(19) United States
(12) Patent Application Publication
(10) Pub. No.: US 2014/0369955 A1
(43) Pub. Date: Dec. 18, 2014

(54) METHODS FOR AUTOLOGOUS STEM CELL TRANSPLANTATION

(71) Applicant: Mayo Foundation for Medical Education and Research, Rochester, MN (US)

(72) Inventors: Svetomir N. Markovic, Rochester, MN (US); Luis F. Porrata, Rochester, MN (US)

(73) Assignee: Mayo Foundation for Medical Education and Research, Rochester, MN (US)

(21) Appl. No.: 14/162,917

(22) Filed: Jan. 24, 2014

Related U.S. Application Data
(63) Continuation of application No. 11/569,653, filed on Apr. 2, 2009, now abandoned.
(60) Provisional application No. 60/575,527, filed on May 28, 2004.

(51) Int. Cl. C12N 5/0783; G01N 33/50; A61K 35/14

(52) U.S. Cl. CPC C12N 5/0646; A61K 35/17; G01N 33/5094; A61K 2035/122
USPC 424/85.2; 424/85.7; 424/85.5; 424/93.71

(57) ABSTRACT

Materials and methods for obtaining populations of lymphocytes and administering the population of lymphocytes to a subject are disclosed herein. In particular, disclosed herein are materials and methods for obtaining lymphocyte populations that contain at least about 0.5×10⁹ NK cells per kilogram weight of the subject from which the cells are harvested.

### CROSS-REFERENCE TO RELATED APPLICATIONS

This application claims priority from U.S. Provisional Application Ser. No. 60/575,527, filed May 28, 2004.

### TECHNICAL FIELD

This document relates to methods and materials for transplantation of autologous lymphocytes.

### BACKGROUND

Autologous stem cell transplantation (ASCT) following chemotherapy has been shown to improve survival in both previously untreated multiple myeloma (MM) and relapsed, chemotherapy-sensitive, aggressive non-Hodgkin's lymphoma (NHL) patients. High relapse rates post-ASCT, however, have been attributed to the inability of high dose therapy (HDT) to eradicate minimal residual disease. In contrast, allogeneic stem cell transplantation following chemotherapy results in lower relapse rates, which have been correlated to early absolute lymphocyte count (ALC) recovery as a manifestation of early graft-versus-tumor effect in the recipient (Kersey et al. (1987) New Engl J Med 317:416; Marmont et al. (1991) Blood 78:2120). Post-allogeneic bone marrow transplant studies have demonstrated that early ALC recovery is associated with prolonged survival (Prowles et al. (1998) Blood 91: 3481). Allogeneic stem cell transplantation has also, however, been associated with a higher incidence of graft-versus-host disease (GVHD).

### SUMMARY

This document provides materials and methods that combine the benefits of ASCT with the benefits of allogeneic stem cell transplantation. The disclosure herein is based in part on the discovery that the total number of lymphocytes, i.e., absolute lymphocyte count (ALC), present in a blood sample taken from a cancer patient any time up to and including day 15 following ASCT is a powerful indicator of prognosis. The disclosure also is based in part on the discovery that the number of natural killer (NK) cells within the transplanted cells can be correlated with the ALC at day 15 after transplant (ALC-15). Thus, the invention relates to materials and methods for treating a mammalian subject (e.g., a human patient) diagnosed with cancer (e.g., breast cancer, non-Hodgkin's lymphoma, multiple myeloma, Hodgkin's disease, or acute myeloid leukemia) with ASCT to achieve an ALC-15 of at least 0.5×10⁹ cells/L of blood. In particular, the invention relates to materials and methods for obtaining autologous cell populations that contain at least 0.5×10⁹ NK cells/kg body weight of the subject from whom the cells are obtained.

In one aspect, this document features a method for treating a patient. The method can include: (a) collecting from the patient a biological sample containing NK cells; (b) monitoring the number of collected NK cells; (c) repeating steps (a) and (b) until the total number of collected NK cells is at least 0.5×10⁹ cells per kg; and (d) returning the collected NK cells to the patient. The biological sample can further contain erythrocytes, and the method can further include returning at least 90% of the erythrocytes to the patient.

The method can further include, prior to returning the collected NK cells to the patient, contacting the collected NK cells with one or more agents that stimulate function or activity of NK cells. The collected NK cells can be retained within a vessel containing the one or more agents. The vessel can contain the one or more agents prior to placement of the NK cells within the vessel. The vessel can have an interior surface, wherein the one or more agents are dispersed on the interior surface. The one or more agents can be in the form of a solid (e.g., a powder). The one or more agents can be selected from the group consisting of IL-2, IL-12, IL-15, IL-17, IL-21, IFN-alpha, and IFN-gamma. The agent can be IL-2 (e.g., at a dose of 1.5 to 2.0 million units).

The method can further include, prior to collecting the biological sample, administering to the patient one or more agents that stimulate NK cell function or activity. The one or more agents can be selected from the group consisting of IL-2, IL-12, IL-15, IL-17, IL-21, IFN-alpha, and IFN-gamma. The one or more agent can be IL-2.

The method can further include, prior to returning the collected NK cells to the patient, subjecting the patient to an immunosuppressive treatment (e.g., radiotherapy, chemotherapy, or surgery with anesthesia). The patient can be diagnosed with cancer (e.g., breast cancer, non-Hodgkin's lymphoma, multiple myeloma, Hodgkin's disease, or acute myeloid leukemia). The patient may be in remission from the cancer prior to collection of the biological sample or prior to return of the collected NK cells.

The method can further include: (f) monitoring the number of NK cells within the patient; and (g) if the number of NK cells in the patient at day 15 is less than 80 NK cells/microliter, administering to the patient one or more agents selected from the group consisting of IL-2, IL-12, IL-15, IL-17, IL-21, IFN-alpha, and IFN-gamma.

Step (b) of the method can further include monitoring the number of collected CD34⁺ cells, step (c) of the method can further include repeating steps (a) and (b) until the total number of collected CD34⁺ cells is at least 2.0×10⁶ cells per kg, and step (d) of the method can further include returning the collected CD34⁺ cells to the patient. The method can further include, prior to collecting the biological sample, administering to the patient one or more agents that can (i) stimulate proliferation of stem cells and/or progenitor cells, and/or (ii) stimulate mobilization of stem cells and/or progenitor cells to the peripheral circulation. The one or more agents can be selected from the group consisting of G-CSF, GM-CSF, SCF, IL-2, IL-7, IL-8, IL-12, and flt-3 ligand.

In another aspect, this document features a method for treating a patient, wherein the method can include: (a) administering autologous lymphocytes to the patient, wherein the autologous lymphocytes are administered in an amount of at least 0.5×10⁹ cells/kg; (b) monitoring the number of NK cells within the patient; and (c) if the number of NK cells at day 15 is less than 80 cells/4, of blood, administering to the patient one or more agents to stimulate NK cell function or activity. The autologous lymphocytes can be removed from the patient. Prior to the removal of the autologous lymphocytes, the patient can be treated with one or more agents selected from the group consisting of IL-2, IL-12, IL-15, IL-17, IL-21, IFN-alpha, and IFN-gamma. Prior to administering the autologous lymphocytes to the patient, the autologous lymphocytes can be contacted in vitro with one or more agents selected from the group consisting of IL-2, IL-12, IL-15, IL-17, IL-21, IFN-alpha, and IFN-gamma. The patient may be diagnosed with cancer (e.g., breast cancer, non-Hodgkin's lymphoma, Hodgkin's disease, multiple myeloma, or acute myeloid leukemia).

In another aspect, the invention features a method for obtaining a population of lymphocytes. The method can include: (a) collecting from a subject a biological sample containing lymphocytes; (b) monitoring the number of NK cells within the collected lymphocytes; and (c) repeating steps (a) and (b) until the total number of NK cells collected from the subject is at least 0.5×10⁹ cells/kg. The method can further include retaining the collected lymphocytes within a vessel that has an identifier corresponding to the subject, and contacting the collected lymphocytes with one or more agents that stimulate NK cell function or activity. The method can further include, prior to collecting the biological sample from the subject, administering to the subject one or more agents to stimulate NK cell function or activity. The one or more agents can be selected from the group consisting of IL-2, IL-12, IL-15, IL-17, IL-21, IFN-alpha, and IFN-gamma.

In still another aspect, the invention features a container containing a population of lymphocytes removed from a subject, wherein the population includes an amount of NK cells that is at least 0.5×10⁹ cells/kg, and wherein the container has an identifier corresponding to the subject. The container can be a blood bag. The container can further contain one or more agents that stimulate NK cell function or activity.

In another aspect, the invention features a container having an inner surface, wherein one or more agents are dispersed on the inner surface, and wherein the one or more agents stimulate NK cell function or activity. The one or more agents can be selected from the group consisting of IL-2, IL-12, IL-15, IL-17, IL-21, IFN-alpha, and IFN-gamma.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a scatter plot showing the correlation between the concentration of infused autograft lymphocytes (A-ALC) and the ALC-15 after autologous peripheral hematopoeitic stem cell transplantation (APHSCT). Spearman's correlation rho factor, r=0.71; P<0.0001.

FIG. 2 is a box plot showing A-ALC in patients with an ALC-15<500 cells/μl and patients with an ALC-15≧500 cells/μl after APHSCT. The horizontal line within each box represents the median, and the lower and upper borders of each box represent the 25^{th} and the 75^{th} percentiles, respectively. Outliers (values that exceed those boundaries) are depicted as single points. By the Wilcoxon rank-sum test, a statistically significant difference was identified when comparing the median value of A-ALC received by patients with an ALC-15<500 cells/μl and the median value of A-ALC received by patients with an ALC recovery≧500 cells/μl after APHSCT (0.34×10⁹ lymphocytes/kg vs. 0.68×10⁹ lymphocytes/kg; P<0.0001).

FIG. 3A is a line graph showing Kaplan-Meier estimates of overall survival of patients infused with an A-ALC<0.50×10⁹ lymphocytes/kg vs. patients infused with an A-ALC≧0.50×10⁹ lymphocytes/kg. The median overall survival was 17 months in the group of patients with an A-ALC<0.50×10⁹ lymphocytes/kg, and 76 months in the group of patients with an A-ALC≧0.50×10⁹ lymphocytes/kg. The overall survival rates at five years were 20 percent and 57 percent, respectively (P<0.0001).

FIG. 3B is a line graph showing Kaplan-Meier estimates of progression-free survival of patients infused with an A-ALC<0.50×10⁹ lymphocytes/kg vs. patients infused with an A-ALC≧0.50×10⁹ lymphocytes/kg. The median progression-free survival was 10 months in the group of patients with an A-ALC<0.50×10⁹ lymphocytes/kg, and 49 months in the group of patients with an A-ALC≧0.50×10⁹ lymphocytes/kg. The progression-free survival rates at five years were 13 percent and 50 percent, respectively (P<0.0001).

FIG. 4 is a scatter plot showing the correlation between peripheral blood absolute lymphocyte count at the time of collection (PC-ALC) and A-ALC. Spearman's correlation rho factor, r=0.76; P<0.0001.

FIG. 5A is a graph showing overall survival of MM patients infused with A-ALC and having an ALC-15≧500 cells/μl vs. those having an ALC-15<500 cells/μl. FIG. 5B is a graph showing progression free survival of MM patients infused with A-ALC and having an ALC-15≧500 cells/μl vs. those having an ALC-15<500 cells/μl. FIG. 5C is a graph showing overall survival of NHL patients infused with A-ALC and having an ALC-15≧500 cells/μl vs. those having an ALC-15<500 cells/μl. FIG. 5D is a graph showing progression free survival of NHL patients infused with A-ALC and having an ALC-15≧500 cells/μl vs. those having an ALC-15<500 cells/μl.

### DETAILED DESCRIPTION

The invention provides materials and methods that combine the benefits of autologous and allogeneic stem cell transplantation. The invention is based in part on the discoveries that ALC-15 can be a powerful indicator of cancer patient prognosis, and that the number of NK cells within a population of transplanted cells can be correlated with ALC-15. Thus, the invention provides materials and methods related to treating a subject having a depleted ALC to achieve an ALC-15 of at least 0.5×10⁹ cells/L (i.e., 500 cells/μl) of blood. In particular, the invention relates to materials and methods for obtaining autologous cell populations that contain at least 0.5×10⁹ NK cells/kg weight of the intended recipient.

### Autologous Stem Cell Transplantation

As used herein, “autologous” as it relates to transplantation refers to a graft in which the donor and recipient is the same individual. Thus, in an autologous transplant cells are harvested from a subject and then returned to the same subject. In contrast, an “allogeneic” transplant refers to a graft in which the donor and recipient are genetically non-identical individuals from the same species. A “xenogeneic” transplant refers to a graft in which the donor and recipient are of different species.

As used herein, an ASCT refers to a procedure in which a sample of a subject's own stem cells are removed and subsequently transplanted back into the same subject. Stem cells can be harvested from bone marrow (BM) or peripheral blood (PB), for example. Once obtained, stem cells can be frozen until needed. For example, stem cells can be obtained from a patient, cryopreserved at temperatures ≦−85° C., and then thawed and returned (i.e., transplanted, typically by transfusion) to the patient. In one embodiment, stem cell aliquots can be thawed, loaded into one or more sterile syringes or infusion bags, and injected intravenously over a period of time ranging from about 30 minutes to about 45 minutes.

In some embodiments, stem cells capable of reconstituting a patient's immune system can be obtained from the patient's peripheral circulation following mobilization of such cells from BM into PB. Mobilization of stem cells can be accomplished by treatment of a patient with one or more factors that can (i) stimulate an increase in proliferation of stem cells and/or progenitor cells, and/or (ii) stimulate migration of stem cells and/or progenitor cells from the BM into the peripheral circulation. Such factors can be administered with adjuvants and/or other accessory substances, separately or in combination as desired. Examples of factors that can be used in this aspect include, without limitation, granulocyte colony-stimulating factor (G-CSF), granulocyte/macrophage colony-stimulating factor (GM-CSF), c-kit ligand (stem cell factor (SCF)), interleukin-2, -7, -8, and -12 (IL-2, IL-7, IL-8, and IL-12), and flt-3 ligand. See, e.g., Bungart et al. (1990) Br. J. Haematol. 76:174; Terella et al. (1993) Bone Marrow Transplant. 11:271; Molineux et al. (1991) Blood 85:275; Grzegorzewski et al. (1994) Blood 83:377; Laterveer et al. (1995) Blood 85:2269; Jackson et al. (1995) Blood 85:2371; and Lyman et al. (1994) Blood 83:2795. Factors to be administered can include, for example, G-CSF alone (e.g., 10 μg/kg/day G-CSF), G-CSF+flt-3 ligand (e.g., 10 μg/kg/day G-CSF+50 μg/kg/day flt-3 ligand), or GM-CSF+flt-3 ligand (e.g., 5 μg/kg/day GM-CSF+50 μg/kg/day flt-3 ligand). See, e.g., Sudo et al. (1997) Blood 89:3186. Such factors can be administered prior to harvest or starting on the day of harvest, for example, and can be given on a daily basis for one to seven days (e.g., for one, two, three, four, five, six, or seven days), or until stem cell harvesting is complete. Factors that stimulate stem cell proliferation or mobilization can be administered using any suitable method. Typically, such factors can be administered parenterally (e.g., by subcutaneous, intrathecal, intraventricular, intramuscular, or intraperitoneal injection, or by intravenous drip). Mobilization of stem cells with, for example, GM-CSF and flt-3 ligand can be evaluated by determining the number of CD34⁺ cells present before, during, and/or after treatment with one or more mobilizing agents. In one embodiment, the number of CD34⁺ cells can be determined by FACS analysis using CD34-specific antibodies conjugated to fluorescent or other labeling moieties.

Following or during mobilization, peripheral blood stem cells (PBSC) can be collected using, for example, an apheresis procedure. The process of apheresis, which is well known in the art, involves removal of whole blood from a patient or donor. Within an instrument that is essentially designed as a centrifuge, the components of the whole blood are separated. One or more of the separated portions is then withdrawn, and the remaining components can be retransfused into the patient or donor. Thus, for example, all or most (e.g., 80%, 90%, 95%, 99%, or 100%) of the erythrocytes in a sample of whole blood can be returned to a patient during an apheresis procedure, while lymphocytes (e.g., NK cells) and stem cells can be collected. Apheresis can be performed as many as four times per week (e.g., one, two, three, or four times per week). In one embodiment, a commercially available blood cell collection device can be used, such as the CS3000® blood cell collection device marketed by the Fenwal Division of Baxter Healthcare Corporation (Fenwal Laboratories, Deerfield, Ill.). Methods for performing apheresis with the CS3000® machine are described in Williams et al. (1990) Bone Marrow Transplantation 5:129-33, and Hillyer et al. (1993) Transfusion 33:316-21, for example, both of which are incorporated herein by reference in their entirety.

Typically, a total blood volume between 9.5 and 10 L per apheresis procedure can be processed at a flow rate of 50 to 70 mL/min. Following collection, a cell count can be performed on an aliquot of the total product to determine the number of stem cells. Cells can be collected until the total sample taken from the patient reaches a concentration of at least 1×10⁶ CD34⁺ stem cells/kg (e.g., at least 2×10⁶ CD34⁺ cell/kg, or at least 3×10⁶ CD34⁺ cells/kg).

Despite various methods of PBSC mobilization, adequate numbers of PBSC for ASCT may be not collected from some patients during a single apheresis procedure. In these patients, BM harvest or a second attempt at PBSC mobilization can be performed. Alternatively, these patients may be excluded from proceeding to ASCT.

Apheresis products can be centrifuged (e.g., at 400 g for 10 minutes), and the plasma can be removed to yield a total volume of, for example, about 100 mL. The resulting cell suspension can be mixed with a physiological freezing solution [e.g., 100 mL minimal essential medium such as MEM-S (Invitrogen Life Technologies, Carlsbad, Calif.) supplemented with 20% dimethylsulfoxide (DMSO)]. Cell/media suspensions can be transferred to freezing bags (such as those manufactured by Delmed, Canton, Mass.) or any other freezing receptacle known in the art, and frozen to −100° C. using, for example, a computer-controlled cryopreservation device (e.g., the Cryoson-BV-6; Cryoson Deutschland GmbH, FRG). The cells then can be transferred into liquid nitrogen and stored at until transplantation.

Patients typically undergo a pre-transplant workup to evaluate, for example, heart, liver, kidney, and lung function, as well as current disease status. In some embodiments, patients deemed to be eligible (e.g., healthy enough) for ASCT are subjected to a tumor debulking procedure prior to ASCT. For example, a patient can be treated with high doses of chemotherapy, radiation therapy, and/or surgery (e.g., surgery with anesthesia) before the transplant. Stem cells for transplant typically are collected prior to tumor debulking regimens, since such potentially lethal procedures can be immunosuppressive and can severely damage or destroy the BM. ASCT following a debulking procedure can reconstitute the patient's immune cells with stem cells present in the transplant.

In some embodiments, a patient's stem cells can be collected by BM harvest using procedures known in the art, or by a stem cell apheresis procedure as described above, for example. Collected stem cells can be cryopreserved, and the patient can undergo a debulking procedure such as high-dose chemotherapy and/or radiation therapy. After the debulking procedure is completed, the patient's stem cells can be transplanted. ASCT can be done almost immediately after a debulking procedure (e.g., 24 to 48 hours after HDT). Alternatively, a longer period of time (e.g., a week to several months) can elapse between a debulking procedure and ASCT. Due to the likelihood of immunosuppression as a result of the debulking procedure, protective isolation precautions generally are taken after ASCT at least until the reinfused stem cells begin to engraft. “Engraftment” refers to a process whereby the transplanted stem cells begin to differentiate into mature blood cells. In addition, stem cells can be treated prior to transplantation with, for example, anticancer drugs or antibodies to reduce the number of cancerous cells that may be present in the sample. Such procedures are referred to as “purging.”

### Absolute Lymphocyte Count

In the methods provided herein, patients are treated by administration of autologous cell populations that can contain stem cells and other cell types, including, for example, RBC and lymphocytes. Lymphocytes are white blood cells (WBC) that are formed in lymphatic tissue throughout the human body (e.g., lymph nodes, spleen, thymus, tonsils, Peyer's Patches, and bone marrow). In normal adults, lymphocytes comprise approximately 22% to 28% of the total number of leukocytes in the circulating blood. As used herein, the term “lymphocyte” includes NK cells, B cells, and T cells (e.g., T helper cells, cytotoxic T cells, and T suppressor cells. NK cells are directly cytotoxic to foreign cells (e.g., foreign cancer cells), and do not require complement activity to effect their lysis. NK cells represent the body's first line of defense against malignancy. B cells produce immunoglobulins, and T cells are involved in modulation of immune responses and in regulation of erythropoiesis. Different types of lymphocytes can be distinguished from each other and from other cell types based on the cell type-specific expression of particular molecular markers, generally cell surface markers. For example, NK cells bear on their surface CD16 and/or CD56 markers. B cells bear at least one of the cell surface markers CD19 and CD20. T cells bear one or more of the cell surface markers CD3, CD4, and CD8. Typically, cytotoxic T cells express CD8, whereas helper T cells express CD4.

As used herein, the term “absolute lymphocyte count” (ALC) refers to the total number of lymphocytes per unit of whole blood or blood cells in a sample or in a subject (e.g., a human patient). A unit can be, for example, a liter (L), milliliter (mL), or microliter (μL). Typically, but not always, ALC is measured as the number of mature lymphocytes per μL of blood, and includes the cumulative numbers of B cells, T cells, and NK cells. Stem cells, lymphocyte precursor cells, and lymphocyte progenitor cells typically are not included in the ALC. Stem cells can be differentiated from lymphocytes in that stem cells express the cell surface marker CD34, whereas mature lymphocytes do not. Moreover, lymphocytes express specific cell surface markers as described above (NK cells: CD16 and/or CD56; B cells: CD20 and/or CD19; T cells: CD3, CD4, and/or CD8), whereas stem cells do not express these markers.

To determine an ALC, a sample of blood can be collected from a patient. For example, blood can be collected in a rubber-stopped tube containing EDTA or another medically acceptable anti-coagulant. Blood can be collected using any route of entry to the circulatory system known in the art. The blood sample then can be analyzed to determine the ALC. In one embodiment, an ALC can be obtained using an automated system for counting blood cells in a sample. Such cell counting systems typically are based on a principle by which unstained, unlabeled cells are sorted and counted based on morphological characteristics including, without limitation, cell size, cell shape, nuclear size, and nuclear shape. For example, the GEN-S™ Hematology Analyzer identifies and counts cell types based on three general criteria: volume, conductivity, and scatter (see U.S. Pat. No. 5,125,737). A blood sample can be treated before analysis with reagents and/or physical agitation to lyse the RBC, thereby leaving WBC for analysis. The Gen-S™ Analyzer uses a process of DC impedance by which the cells are collided with light to physically measure the volume displaced by the entire cell in an isotonic diluent. Cell size thus can be accurately determined regardless of the orientation of the cell in the light path. Cells can be further collided with an alternating current in the radio frequency range that can permeate cell membranes, such that information can be obtained with regard to internal structure including, for example, chemical composition and nuclear structure. A cell can be collided with a laser beam that, upon contacting the cell, scatters and spreads out in all directions, generating median angle light scatter signals. These signals can be collected to yield information regarding cellular granularity, nuclear lobularity, and cell surface structure. Thus, such a system can count and differentiate RBC from WBC based on the presence or absence of a nucleus, and can count and differentiate the different types of WBC based on the ratio of nuclear to cytoplasmic volume, lobularity of the nucleus, and granularity of the cytoplasm as described below, for example.

ALC also can be determined by placing a known volume of a blood sample onto a glass microscope slide, smearing the sample to create a thin film of blood on the slide, and staining the sample using standard histological stains such as, for example, hematoxylin and eosin (H & E). Briefly, a blood smear can be dried and subsequently fixed onto a slide using a fixative such as, without limitation, neutral buffered formalin, formaldehyde, paraformaldehyde, glutaraldehyde, Bouin's solution, mercuric chloride, or zinc formalin. The slides then can be immersed in a solution of Harris Hematoxylin, rinsed in water, immersed in a solution of Eosin, rinsed in water, dehydrated in ascending alcohol solutions, and cleared in xylenes. In blood smears that have been stained using H & E, nuclei and other basophilic structures stain blue, whereas cytoplasm and other acidophilic structures stain light to dark red (Sheehan et al. (1987) Theory and Practice of Histotechnology, 2nd Edition, Battelle Memorial Institute, Columbus, Ohio), which is incorporated herein by reference in its entirety. The number of lymphocytes present in a blood smear can be counted based on lymphocytic morphological criteria accepted in the art.

For example, when stained with H & E, the lymphocyte nucleus is deeply colored (purple-blue) and is composed of dense aggregates of chromatin within a sharply defined nuclear membrane. The nucleus generally is round, eccentrically located, and surrounded by a small amount of light blue staining cytoplasm. The volume of nucleus to cytoplasm in a lymphocyte typically is about 1:1.2. Lymphocytes can be differentiated from RBC in that RBC have no nuclei. Lymphocytes can be differentiated from neutrophils in that neutrophils have nuclei with 2 to 5 lobes, while lymphocyte nuclei are not lobed. Lymphocytes can be differentiated from basophils and eosinophils in that those cells have cytoplasmic granules, while lymphocytes do not have cytoplasmic granules. Lymphocytes can be differentiated from monocytes in that monocytes are 16 to 20 μm in diameter, while lymphocytes are 7 to 10 μm in diameter. In addition, one of skill in the art may refer to any of a number of hematology or histological texts or atlases (e.g., Wheater et al. (1987) Functional Histology 2nd Ed. Churchill Livingstone, incorporated herein by reference in its entirety) to determine the physical appearance of a lymphocyte.

ALC also can be determined by immunolabeling lymphocytes with antibodies specific for lymphocyte cell surface markers, and counting the immunolabeled cells using fluorescence flow cytometry (FFC). For example, NK cells can be labeled with one or more fluorescently labeled antibodies specific for CD16 and/or CD56. Similarly, B cells can be labeled with one or more fluorescently labeled antibodies specific for the adhesion molecules CD20 and/or CD19, and T cells can be labeled with one or more fluorescently labeled antibodies specific for CD3, CD4, and/or CD8, and. To determine ALC, cell surface marker-specific antibodies can be labeled with the same fluorophore (e.g., Cy-5, fluorescein, or Texas Red). In a FFC procedure, individual cells are held within a thin stream of fluid and passed through one or more laser beams, one cell at a time, causing light to scatter and the fluorescent dyes to emit light at various predetermined frequencies. Photomultiplier tubes convert the light to electrical signals, allowing for quantitation of the number of cells bearing the fluorophore. If all lymphocyte subtypes are labeled with the same fluorophore, quantification of the number of fluorophore-bearing cells will yield an ALC. FFC and quantitation is further described in, for example, U.S. Pat. No. 4,499,052. In addition, a FFC machine can be adapted for fluorescence activated cell sorting (FACS), i.e., the separation (and collection) of (a) fluorescent cells from non-fluorescent cells; (b) strongly fluorescent cells from weakly fluorescent cells; or (c) cells fluorescing at one wavelength from cells fluorescing at another wavelength.

An ALC-15 of at least 500 cells/μL of blood has been correlated with increased survival of patients following tumor debulking and ASCT. In the methods provided herein, patients (e.g., cancer patients undergoing ASCT) can be treated to achieve an ALC-15 of at least 500 cells/μL. As used herein an “ALC-15” refers to an ALC determined any time up to and including day 15 following ASCT. “Day 15” refers to a 15 day period of time where day 1 is the day following completion of an ASCT. Thus, a “day 15” blood sample can be obtained anytime within the first 360 hours after completion of an ASCT (i.e., post-ASCT) but not more than 384 hours after completion of the ASCT. For example, a “day 15” sample can be obtained 1, 2, 3, 4, 5, 6, 7, 8, 9, 10, 11, 12, 13, 14, or 15 days after completion of an ASCT. Samples obtained any time between 3 to 15 days, 5 to 15 days, or 8 to 15 days following completion of an ASCT can be particularly useful. Completion of an ASCT occurs at that time when all of the stem cells intended for transplant have been administered to the patient.

### Methods for Obtaining Populations of Cells and Treating Patients

The number of NK cells in a transplanted population of cells can be correlated with ALC-15. Thus, the invention provides methods that can be used to obtain a population of cells containing lymphocytes, as described above, wherein the population contains a particular number of NK cells. For example, the methods provided herein can include the following steps: (a) collecting from a patient a biological sample (e.g., a blood sample) containing NK cells, (b) monitoring the number of NK cells in the collected sample, and (c) repeating steps (a) and (b) until the total number of collected NK cells is at least about 0.5×10⁹ cells/kg weight of the patient (e.g., 0.48×10⁹ cells/kg, 0.49×10⁹ cell/kg, 0.50×10⁹ cells/kg, 0.51×10⁹ cells/kg, or 0.52×10⁹ cells/kg). The patient can be a human cancer patient diagnosed with, for example, non-Hodgkin's lymphoma, Hodgkin's disease, multiple myeloma, acute myeloid leukemia, or breast cancer. The methods provided herein also can include the step of returning the collected NK cells to the patient. Typically, the cell population can be returned to the patient by intravenous infusion, although any suitable method known in the art can be used. In some embodiments, the patient can be in remission from the cancer, either prior to collection of the cells or prior to returning the cells to the patient.

NK cells can be collected using an apheresis procedure as described above. In addition, the number of collected NK cells can be monitored. For example, the number of NK cells can be determined at one or more points during collection of the sample from the patient. The number of NK cells also can be determined after completion of a collection. Once the population of total collected cells includes at least about 0.5×10⁹ NK cells/kg, they can be returned to the patient. The number of collected NK cells can be monitored using methods such as those described above. In some embodiments, the number of collected NK cells can be determined using immunolabeling with one or more fluorescently labeled antibodies specific for CD16 and/or CD56, and counting with FACS.

In addition to monitoring the number of NK cells collected from a patient, the methods provided herein also can include monitoring the number of CD34⁺ cells collected from the patient. In one embodiment, for example, a method can include (a) collecting from a patient a biological sample containing NK cells and CD34⁺ cells, (b) monitoring the number of collected NK cells and CD34⁺ cells, and (c) repeating steps (a) and (b) until the total number of collected NK cells is at least 0.5×10⁹ cells per kg and the total number of collected CD34⁺ cells is at least about 2.0×10⁶ cells/kg. The numbers of collected NK and CD34⁺ cells can be determined as described herein, for example. The method also can include the step of returning the collected cells to the patient.

The methods provided herein also can include treatment of a patient or a cell population (e.g., in a biological sample such as an apheresis product) with one or more agents that stimulate proliferation, maturation, differentiation, function, and/or activity of immune cells (e.g., NK cells). For example, NK cells in a patient or in a biological sample can be contacted with an agent such as IL-2, IL-12, IL-15, IL-17, IL-21, interferon alpha (IFN-α), or interferon gamma (IFN-γ). These agents can be native factors obtained from a natural source, factors produced by recombinant DNA methodology, chemically synthesized polypeptides or molecules, or any derivative having the functional activity of the native factor. Since agents such as these can enhance the number and/or activity of NK cells, a patient may be subjected to shorter or fewer apheresis procedures in order to harvest a cell population containing at least about 0.5×10⁹ cells/kg.

In one embodiment, a population of cells (e.g., a population of collected autologous lymphocytes containing NK cells) can be contacted in vitro with one or more agents such as those listed above. For example, collected cells can be placed in a vessel (e.g., a bag, a tube, a vial, or any other suitable container) and contacted with one or more agents such as those described above. In one embodiment, NK cells can be contacted in vitro with IL-2 at a dose of, for example, about 1.5×10⁶ to about 2.0×10⁶ units. NK cell enhancing agents can be added to cells within a container such as a bag (e.g., a blood bag), tube, or vial, or such a vessel can contain one or more such agents prior to placement of cells within the vessel. In some embodiments, one or more agents can be dispersed on an inner surface of the vessel. For example, an agent in liquid form can be dispersed (e.g., sprayed) onto an inner surface of the vessel and allowed to dry. Alternatively, an agent in solid (e.g., lyophilized or powdered) form can be dispersed on an inner surface of the vessel. In another alternative, an agent in liquid or solid form can simply be placed within the vessel.

Alternatively, one or more NK cell enhancing agents such as those listed above can be administered to a patient. A patient can be treated with such an agent prior to collection of a biological sample containing NK cells, or a patient can be treated post-ASCT. For example, the number of NK cells in the PB of a patient can be monitored following ASCT, and an NK cell enhancing agent can be administered to the patient if the number of NK cells is less than a particular threshold at a particular time point (e.g., at post-transplant day 15). A suitable threshold can be, for example, about 80 NK cells/μL of blood (e.g., about 75 NK cells/μL or about 85 NK cells/μL). Similarly, an NK cell enhancing agent can be administered to a patient post-ASCT if the ALC-15 is less than 500 cells/μL of blood. Agents such as those listed above can be administered to a patient via any pharmaceutically acceptable route known in the art, including, for example, intravenous injection, intra-arterial injection, subcutaneous injection, intramuscular injection, intraperitoneal injection, or oral administration in the form of a tablet, capsule, or syrup. In one embodiment, IL-2 can be administered to a patient prior to collection of NK cells or after ASCT. In another embodiment, a patient can be treated with IFN-γ at a concentration of, for example, between about 1×10⁵ and about 1×10² units/m². When the treatment is post-ASCT, the agent(s) can be administered from the day of transplant up to about 21 days following the transplant.

Patients or biological samples containing NK cells and other lymphocytes also can be treated with one or more agents that activate the T cell signal transduction pathway, leading to lymphocyte activation. A T cell activator can be, without limitation, one or more of the following: IL-1, IL-2, IL-4, IL-5, IL-6, IL-7, IL-12, IL-13, IFNα, IFNγ, tumor necrosis factor (TNFα), an anti-CD3 antibody or antigen-binding fragments thereof (anti-CD3), an anti-CD28 antibody or antigen-binding fragments thereof (anti-CD28), phytohemagglutinin, concanavalin-A, and phorbol esters. As above, these agents can be native factors obtained from a natural source, factors produced by recombinant DNA methodology, chemically synthesized polypeptides or molecules, or any derivative having the functional activity of the native factor.

### Containers of Lymphocytes

The invention also provides vessels containing a population of lymphocytes. Suitable containers include, for example, bags (e.g., blood bags), tubes, vials, and the like. Typically, the lymphocyte population has been removed from a subject (e.g., a human) diagnosed with cancer. The population of cells within a container can include at least about 0.5×10⁹ NK cells/kg weight of the subject from which they were removed. The container also can have an identifier (e.g., a label) corresponding to the subject, so that a practitioner such as a clinician or a technician can determine that the cells within the container were obtained from a particular individual. In addition, a vessel can contain one or more agents that stimulate NK cell proliferation, maturation, differentiation, function, and/or activity. For example, a vessel can contain IL-2, IL-12, IL-15, IL-17, IL-21, IFN-α, and/or IFN-γ.

In another embodiment, the invention provides containers (e.g., bags such as blood bags, tubes, vials, and the like) having an inner surface with one or more NK cell enhancing agents dispersed thereon. For example, a container can have an agent such as IL-2, IL-12, IL-15, IL-17, IL-21, IFN-α, or IFN-γ dispersed on an inner surface. The agent(s) can be in a liquid solution and sprayed onto an inner surface of a container, or the agent(s) can be in a solid (e.g., powdered or lyophilized) form and dispersed onto an inner surface of the container.

The invention will be further described in the following examples, which do not limit the scope of the invention described in the claims.

### EXAMPLES

### Example 1

### A-ALC is Positively Correlated with ALC-15

### Subjects

One hundred and ninety non-Hodgkin's lymphoma patients that received autologous peripheral blood stem cell transplantation were included in this study. Patients that received bone marrow harvest or a combination of autologous peripheral blood stem cell transplantation and bone marrow harvest were excluded. This was a retrospective study in which data were prospectively collected over time and entered into a computerized database. Response to therapy, relapse, and survival data were updated continuously. No patients were lost to follow-up.

End Points:

The primary end point of the study was the correlation between the number of infused A-ALC and ALC-15. Secondary end points included overall survival and progression-free survival based on the dose of infused A-ALC, as well as assessment of factors impacting on A-ALC. The ALC-15 was calculated from the standard complete blood cell count, and the infused A-ALC for each apheresis unit collection was calculated as follows: (% collection lymphocytes)×(absolute WBC)/kg.

Prognostic Factors:

The international age-adjusted prognostic index [age (≧60 vs. <60), LDH >normal for age/sex, performance status (PS; ≧2 vs. <2), extranodal sites (≧2 vs. <2), and stage (I/II vs. III/IV] at the time of transplantation, in addition to the number of pretransplant treatments, chemo-sensitive disease status, and complete response (CR) status before transplantation were used in the study.

Peripheral Blood Stem Cell (Lymphocyte Autograft) Collection:

Patients received granulocyte-colony stimulating-factor (G-CSF; 10 μg/kg) daily for 5-7 consecutive days by subcutaneous injection. Apheresis collections were performed with a Fenwal CS3000-plus blood-cell collector (Baxter, Deerfield, Ill.). Ten to twelve liters of blood were processed daily, at flow rates of 50-60 ml/min using Hickman catheter or antecubital veins. Patients underwent daily apheresis collections until a target of 2.0×10⁶ CD34 cells/kg or greater was achieved. Pre-stem cell mobilization ALC was obtained from a complete blood cell count prior to G-CSF administration. A peripheral blood absolute lymphocyte count at the time of collection (PC-ALC) was obtained from a complete blood cell count.

Conditioning Regimens:

Ninety-six patients received BEAM [BCNU (300 mg/m²), etoposide (100 mg/m²), ARA-C (100 mg/m²), and melphalan (140 mg/m²)]; 82 patients received BEAC [BCNU (300 m g/m²), etoposide (100 mg/m²), ARA-C (100 mg/m²), and cyclophosphamide (35 mg/kg)]; and 12 patients received cyclophosphamide (60 mg/m²) and total body irradiation (12 Gy).

Response and Survival:

Response criteria were based on the guidelines from the non-Hodgkin's lymphoma International Workshop (Cheson et al. (1999) J. Clin. Oncol. 17:1244-1253). Complete response (CR) was defined as complete regression of all measurable or evaluative disease, including radiologically demonstrable disease, BM involvement, or PB involvement. Partial response (PR) was defined as a reduction in the sum of the products of measurable lesions' longest diameter and perpendicular diameters of 75% or greater, with a 50% or greater decrease in hepatomegaly or splenomegaly (measured from the costal margin), if there was previous known liver or spleen involvement. Stable disease was defined as less than PR but is not progressive disease. Disease progression was defined as a 50% or more increase in the sum of the products of the longest diameter and its perpendicular diameter of measurable lesion(s) from the prestudy measurement, the appearance of new lesions, or a 2-cm increase in spleen or liver size due to lymphoma. Relapsed disease was defined as the appearance of any new lesion or increase by 50% or more in the size of previously involved sites. Overall survival was measured from the date of transplantation to the date of death or last follow-up. Progression-free survival was defined as time from transplantation to disease progression, relapse, death, or last follow-up.

Statistical Analysis:

Overall survival (OS) and progression-free survival (PFS) were analyzed using the method described by Kaplan and Meier ((1958) J. Am. Stat. Assoc. 53:457-481). Differences between survival curves were tested for statistical significance using the 2-tailed log-rank test. The Cox proportional hazards model ((1972) J. R. Stat. Soc. 34:187-202) was used to assess A-ALC, as a prognostic factor for posttransplant OS and PFS times as well as to adjust for other known prognostic factors. Risk ratios reported are for risks associated with patients having high (≧0.5×10⁹ lymphocytes/kilogram) versus low (<0.5×10⁹ lymphocytes/kilogram) A-ALC values. Prognostic factors tested included age (60 years or older), LDH (greater than normal for age/sex), cancer stage (III/IV), extranodal sites (2 or more), performance status (ECOG, 2 or greater), number of pretransplant treatments regimens, chemosensitive disease defined as CR or PR, and CR status alone before transplantation. Factors tested to identify association with ALC-15 (as a continuous variable) included A-ALC, age (60 or greater), conditioning regimens, CR status pre-transplantation, disease status prior to transplantation (relapse, progression, PR, or CR), extranodal sites (2 or more), histology, LDH (greater than normal for age/sex), number of pre-transplant treatment regimens, performance status (2 or more), posttransplant cytokines (G-CSF vs. GM-CSF), pre-mobilization ALC, sex, and stage III/IV. Factors tested to identify association with A-ALC (as a continuous variable) included age (60 or more), CR status pre-transplantation, disease status pre-transplantation (relapse, progression, PR, or CR), extranodal sites (2 or more), histology, LDH (greater than normal for age/sex), number of pre-transplant treatment regimens, performance status (2 or more), PC-ALC, pre-mobilization ALC, sex, and stage III/IV. The cutoff of an ALC of 500 cells/μl or more at day 15 after APHSCT was used based on previous publications (Porrata et al. (2002) 16:1311-1318; Porrata et al. (2001) Bone Marrow Transplantation 28:865-871; Nieto et al. (2003) Biol. Blood Marrow Trans. 9:72 (abstract #30); and Porrata et al. (2002) Brit. J. Haematol. 117:629-633). The cutoff of an infused A-ALC of 0.50×10⁹ lymphocytes/kilogram was based on the median of the infused A-ALC for the cohort group. This choice of threshold yielded the greatest differential in survival at 0.5×10⁹ lymphocytes/kilogram, based on χ² values analyzed at different cut-points (0.2, 0.25, 0.3, 0.35, 0.4, 0.45, 0.5, 0.55, 0.6, 0.65, 0.7, 0.75, 0.8, 0.85, and 0.9×10⁹ lymphocytes/kilogram) from log-rank tests. Chi-square analysis and Fisher Exact tests were used to determine relations between categorical variables; Wilcoxon rank-sum test and Spearman correlation coefficient were used for continuous variables. All P values represented were 2-sided, and statistical significance was declared at P<0.05.

Patient Characteristics:

For the 190 patients evaluated in the study; the median age for the cohort group was 54 years (range, 23-73 years) at the time of transplantation. The median infused autograft absolute lymphocyte count was 0.5×10⁹ lymphocytes/kilogram (range, 0.008-2.34×10⁹ lymphocytes/kilogram). Patient baseline characteristics are listed in Table 1 according to patients that received an A-ALC<0.5×10⁹ lymphocytes/kilogram versus patients that received ≧0.5×10⁹ lymphocytes/kilogram. No differences between the groups were identified for the patient characteristics or prognostic factors, except for ALC at day 15 post-APHSCT. None of the patients received purged or CD34-selected stem cells.

Role of Infused Autograft Lymphocytes on ALC-15:

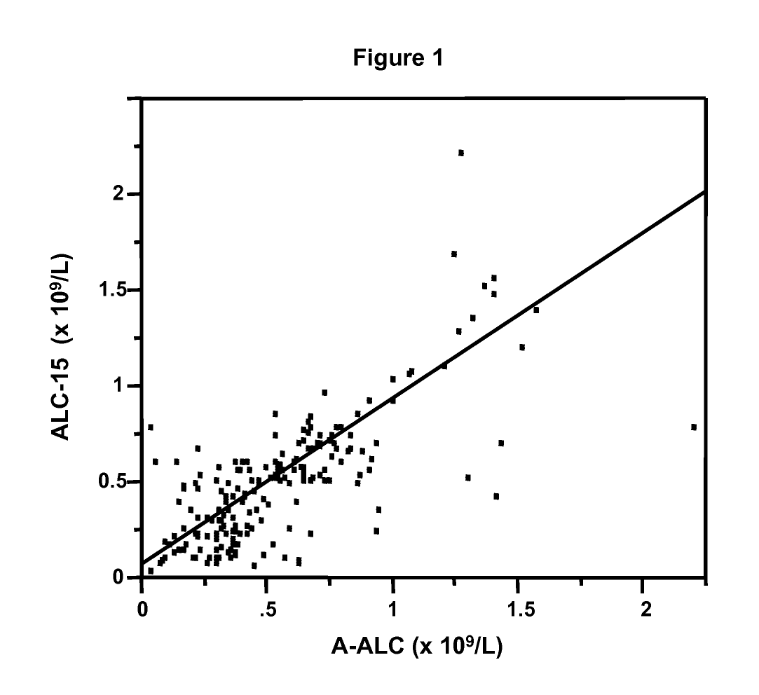

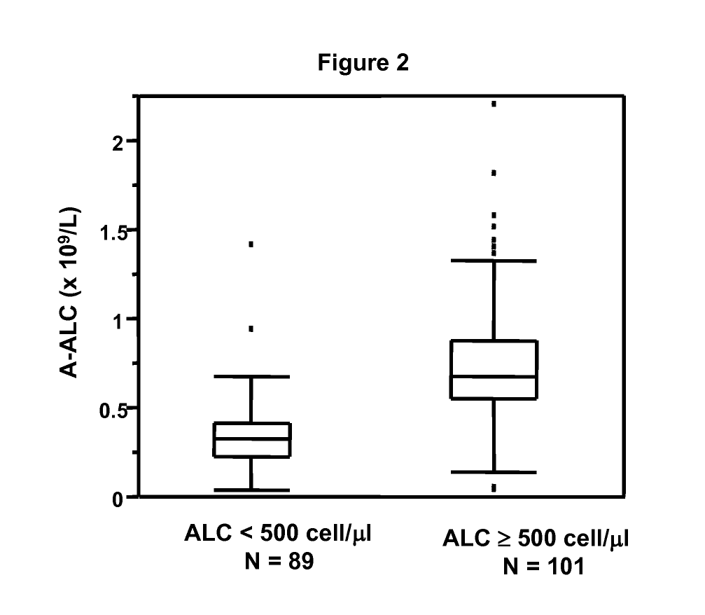

As shown in Table 2, there was a strong correlation between the infused A-ALC and ALC-15 (Spearman's rho, r=0.71, P<0.0001; FIG. 1). Stratifying patients with ALC-15 of ≧500 cells/μl compared with those with ALC-15<500 cells/μl revealed that a higher median number of lymphocytes was infused into patients achieving an ALC-15≧500 cells/μl compared with those with ALC-15<500 cells/μl [median number of 0.68×10⁹ lymphocytes/kilogram (range 0.04-2.21×10⁹ lymphocytes/kilogram), vs. 0.34×10⁹ lymphocytes/kilogram (range 0.04-1.42×10⁹ lymphocytes/kilogram), P<0.0001; FIG. 2]. The mean number of A-ALC infused into patients with ALC-15≧500 cells/μl was 0.75×10⁹ lymphocytes/kilogram (95% CI: 0.69-0.81×10⁹ lymphocytes/kilogram), compared with 0.36×10⁹ lymphocytes/kilogram for patients with ALC-15<500 cells/μl (95% CI: 0.30-0.42×10⁹ lymphocytes/kilogram).

TABLE 1
Baseline Characteristics of Patients According to A-ALC

| | Infused A-ALC | Infused A-ALC | |
|---|---|---|---|
| | < 0.5 × 10⁹ | ≧ 0.5 × 10⁹ | P-value |
| | lymphocytes/kg* | lymphocytes/kg* | between |
| Characteristic | (N = 96) | (N = 94) | groups |
| Median Age (yr) | 54 | 54.6 | 0.74 |
| Gender | | | |
| Female | 39 | 35 | 0.74 |
| Male | 57 | 59 | 0.63 |
| Histology (REAL classification) | | | 0.32 |
| Diffuse large cell | 52 | 63 | |
| Mantle cell | 11 | 10 | |
| Follicular large cell | 12 | 6 | |
| Follicular | 8 | 5 | |
| Anaplastic T cell | 3 | 4 | |
| T-rich B cell | 4 | 2 | |
| T cell lymphoma | 2 | 2 | |
| Burkitts | 1 | 0 | |
| Angiocentric T cell | 1 | 1 | |
| Angiocentric B cell | 0 | 1 | |
| Anaplastic B cell | 1 | 0 | |
| Lymphoblastic | 1 | 0 | |
| lymphoma | | | |
| Disease status at transplantation | | | 0.20 |
| First relapse | 4 | 2 | |
| Second relapse | 0 | 2 | |
| Progression | 2 | 2 | |
| Partial response | 74 | 75 | |
| Complete response | 16 | 13 | |
| Prognostic factors for NHL | | | |
| at time of transplantation | | | |
| Age ≧ 60 | 33 | 28 | 0.50 |
| LDH normal for | 27 | 31 | 0.47 |
| age/sex | | | |
| Performance status < 2 | 92 | 89 | 0.41 |
| Extranodal sites < 2 | 93 | 89 | 0.78 |
| Stage III/IV | 69 | 62 | 0.83 |
| Number of pre-transplant | 70 | 75 | 0.25 |
| regimens ≦ 2 | | | |
| Post-transplant cytokines | | | |
| GM-CSF | 41 | 47 | 0.28 |
| G-CSF | 55 | 47 | 0.31 |
| ALC-15 | | | <0.0001 |
| ≧500 cells/μl | 11 | 78 | |
| <500 cells/μl | 85 | 16 | |

*This choice of threshold yielded the greatest differential in survival at 0.5 × 10⁹ lymphocytes/kg based on χ² analyzed at different cut-points (0.2 to 0.9 × 10⁹ lymphocytes/kg) from log-rank tests.

There was no correlation between A-ALC and the other baseline characteristics and prognostic factors listed in Table 2. In addition, there was no correlation between A-ALC and ALC recovery at 6 months post-APHSCT (r=0.08, P=0.25).

TABLE 2
Correlation between ALC-15 and patients characteristics/prognostic factors

| Characteristic/prognostic factor | P value |
|---|---|
| A-ALC | <0.0001 |
| CD34 cell dose | 0.92 |
| Clinical status pre-transplantation | 0.66 |
| Conditioning regimens | 0.23 |
| CR status pre-transplantation | 0.80 |
| Post-transplant G-CSF | 0.55 |
| Post-transplant GM-CSF | 0.36 |
| Histology | 0.51 |
| International prognostic index at transplantation | |
| Age (≧60 vs. <60) | 0.47 |
| Extranodal site (≧2 vs. <2) | 0.91 |
| LDH (normal vs. >normal for age/sex) | 0.26 |
| Performance status (≧2 vs. <2) | 0.36 |
| Stage (I/II vs. III/IV) | 0.55 |
| Number of pre-transplant treatment regimens | 0.91 |
| Pre-mobilization ALC | 0.40 |
| Sex | 0.24 |

Survival Based on the Infused A-ALC:

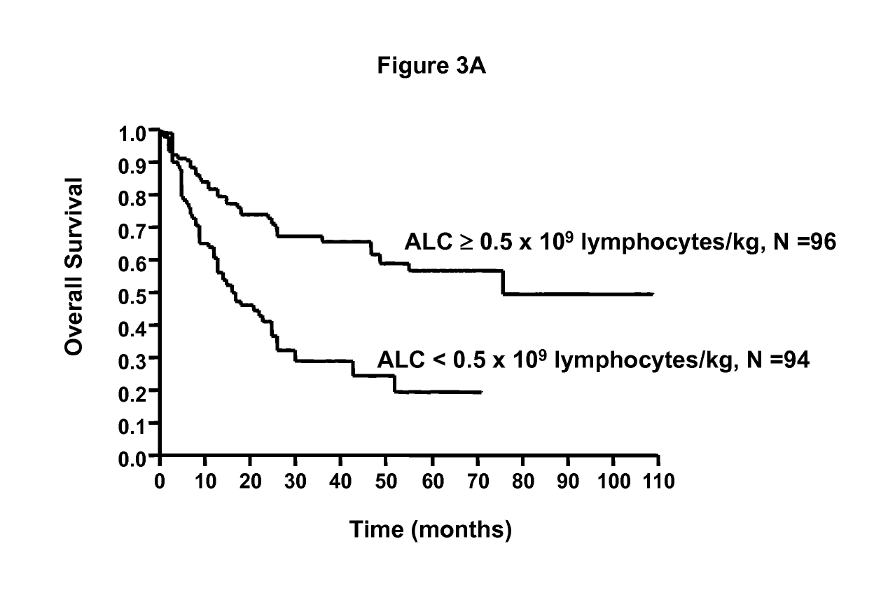

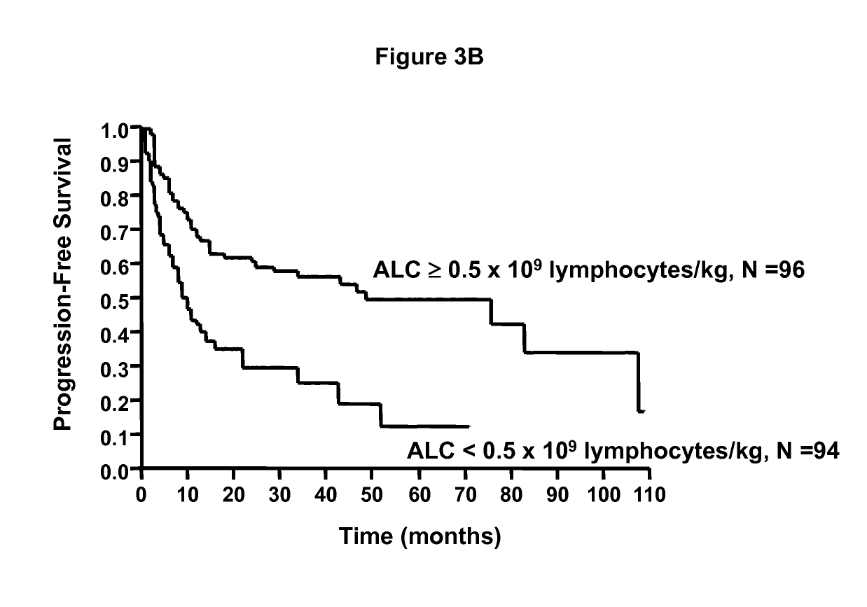

By December 2001, 95 (50%) of the 190 patients in the study had died. Recurrent or progression of disease was the cause of death in 86 patients. The transplant related mortality for the cohort group was only 4.7% (9/190). Three patients died of complications of myelodysplastic syndrome, two patients of acute respiratory distress syndrome, one patient of leukemia, one patient of pneumonia, one patient of renal failure, and one patient of septic shock. None of the patients developed clinically evident autologous graft-versus-host disease. The median follow-up time for all patients was 36 months, with a maximum of 111 months. Of the 86 deaths due to disease relapse or progression, 31 (36%) patients had an infused autograft ALC of ≧0.5×10⁹ lymphocytes/kilogram, and 55 (64%) patients had an infused autograft ALC<0.5×10⁹ lymphocytes/kilogram. Of the 9 death cases due to transplant related mortality, (56%) patients received an infused autograft ALC of ≧0.5×10⁹ lymphocytes/kilogram, while 4 (44%) patients had an infused autograft ALC<0.5×10⁹ lymphocytes/kilogram. Using the cut-off point of 0.5×10⁹ lymphocytes/kilogram, the median overall survival (FIG. 3A) and progression-free survival (FIG. 3B) times were significantly better for patients infused with autograft ALC of ≧0.50×10⁹ lymphocytes/kilogram, compared with patients infused with autograft ALC<0.50×10⁹ lymphocytes/kilogram (76 vs. 17 months, P<0.0001; 49 vs. 10 months, P<0.0001, respectively).

Because of the multiple histological diagnoses, the effect of the lymphocyte dose on the OS and PFS was assessed in patients with diffuse large cell lymphoma and follicular lymphoma, the two largest histological subgroups in the study. Using a cut-off point of 0.50×10⁹ lymphocytes/kilogram, the median OS and PFS were significantly better for patients infused with A-ALC≧0.50×10⁹ lymphocytes/kilogram compared with patients infused with A-ALC<0.50×10⁹ lymphocytes/kilogram in the diffuse large cell group (55 vs. 16 months, P<0.0063; 49 vs. 9 months, P<0.0067, respectively) and in the follicular group (not reached vs. 9 months, P<0.0001; 108 months vs. 7 months, P<0.0001, respectively).

Univariate Analysis:

Age, chemosensitive disease, CR status before transplantation, number of pre-transplantation chemotherapy regimens, and stage were not predictive of OS and PFS. A-ALC, extranodal sites, LDH, and performance status were significant predictors of OS in the univariate analysis. Only A-ALC and LDH were significant predictors in the univariate analysis for PFS (Table 3).

Multivariate Analysis:

A-ALC was an independent predictor for OS (RR=0.60; P<0.0001) and PFS (RR=0.64; P<0.0001) when compared to the significant predictors identified in the univariate analysis, including extranodal sites, LDH, and performance status (Table 4).

TABLE 3
Univariate analysis for overall survival and progression-free survival

| Prognostic | | | | | | |
|---|---|---|---|---|---|---|
| factors at | Overall survival | | | Progression-free surv. | | |
| transplantation | RR^{a} | 95% CI | P | RR | 95% CI | P |
| Age | 1.12 | 0.90-1.38 | 0.29 | 1.09 | 0.89-1.32 | 0.42 |
| ≧60 vs. <60 | | | | | | |
| A-ALC ≧0.5 × | 0.60 | 0.48-0.75 | <0.0001 | 0.64 | 0.52-0.78 | <0.0001 |
| 10⁹ vs. | | | | | | |
| A-ALC <0.5 × | | | | | | |
| 10⁹ | | | | | | |
| Chemosensitive | 0.56 | 0.13-1.18 | 0.15 | 0.79 | 0.31-1.46 | 0.47 |
| disease | | | | | | |
| (CR^{b} + PR^{c}) | | | | | | |
| CR status | 0.95 | 0.70-1.24 | 0.72 | 1.02 | 0.78-1.29 | 0.88 |
| before | | | | | | |
| transplantation | | | | | | |
| Extranodal | 0.56 | 0.38-0.91 | 0.02 | 0.67 | 0.45-1.30 | 0.12 |
| sites | | | | | | |
| <2 vs. ≧2 | | | | | | |
| LDH > normal | 1.30 | 1.05-1.60 | 0.02 | 1.22 | 1.05-1.49 | 0.05 |
| Performance | 0.60 | 0.42-0.92 | 0.02 | 0.71 | 0.49-1.14 | 0.14 |
| status | | | | | | |
| <2 vs. ≧2 | | | | | | |
| No pre- | 1.00 | 0.77-1.28 | 0.99 | 1.03 | 0.81-1.29 | 0.80 |
| transplant | | | | | | |
| chemotherapy | | | | | | |
| regimens | | | | | | |
| Stage I/IV | 0.90 | 0.70-1.11 | 0.31 | 0.91 | 0.74-1.12 | 0.40 |
| vs. III/IV | | | | | | |

^{a}RR = relative risk;
^{b}CR = complete response;
^{c}PR = partial response

TABLE 4
Multivariate analysis for overall survival and progression-free survival

| Prognostic | | | | | | |
|---|---|---|---|---|---|---|
| factors at | Overall survival | | | Progression-free surv. | | |
| transplantation | RR^{a} | 95% CI | P | RR | 95% CI | P |
| A-ALC ≧0.5 × | 0.60 | 0.48-0.75 | <0.0001 | 0.64 | 0.53-0.78 | <0.0001 |
| 10⁹ vs. | | | | | | |
| A-ALC <0.5 × | | | | | | |
| 10⁹ | | | | | | |
| LDH > normal | 1.21 | 0.97-1.52 | 0.09 | 1.22 | 1.00-1.48 | 0.06 |
| Extranodal | 0.76 | 0.50-1.26 | 0.26 | | | |
| sites | | | | | | |
| <2 vs. ≧2 | | | | | | |
| Performance | 0.70 | 0.47-1.13 | 0.14 | | | |
| status | | | | | | |
| <2 vs. ≧2 | | | | | | |

^{a}RR = relative risk; likelihood ratio, P < 0.0001

Autograft Peripheral Blood Absolute Lymphocyte Count:

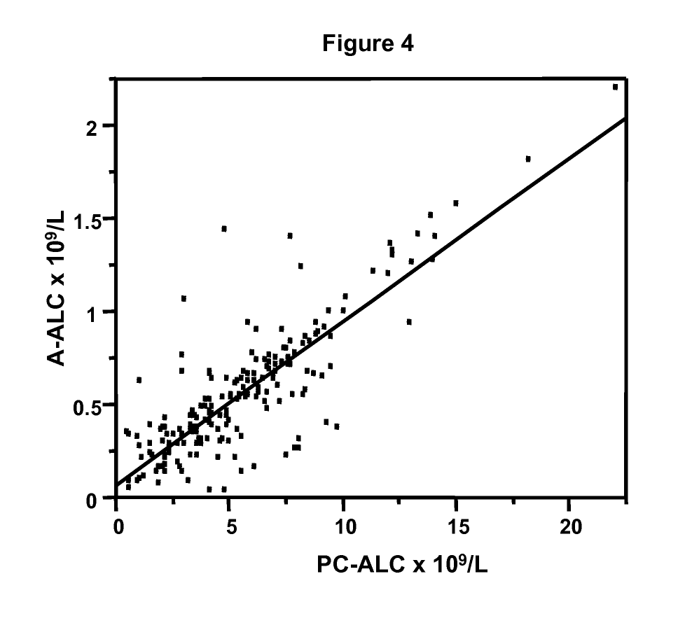

Factors influencing A-ALC collection were investigated. As shown in Table 5 and FIG. 4, there was a strong correlation between PC-ALC and A-ALC (r=0.76, P<0.0001). Patient clinical characteristics and disease status did not show any impact on PC-ALC (Table 6). Because all patients received the same stem cell mobilization regimen (G-CSF), this factor was not included in the analysis. There was no association between A-ALC and the other patient baseline characteristics and prognostic factors listed in Table 5.

TABLE 5
Correlation between A-ALC and patients
characteristics/prognostic factors

| Characteristics/prognostic factors | P value |
|---|---|
| PC-ALC | <0.0001 |
| CD34 cell dose | 0.80 |
| Clinical status pre-transplantation | 0.44 |
| CR status pre-transplantation | 0.35 |
| Histology | 0.45 |
| International prognostic index at transplantation | |
| Age (≧60 vs. <60) | 0.73 |
| Extranodal site (≧2 vs. <2) | 0.54 |
| LDH (normal vs. > normal for age/sex) | 0.19 |
| Performance status (≧2 vs. <2) | 0.33 |
| Stage (I/II vs. III/IV) | 0.98 |
| Number of pre-transplant treatment regimens | 0.21 |
| Pre-mobilization ALC | 0.70 |
| Sex | 0.45 |

TABLE 6
Correlation between PC-ALC and patients
characteristics/prognostic factors

| Characteristics/prognostic factors | P value |
|---|---|
| Clinical status pre-transplantation | 0.24 |
| CR status pre-transplantation | 0.15 |
| Histology | 0.30 |
| International prognostic index at transplantation | |
| Age (≧60 vs. <60) | 0.30 |
| Extranodal site (≧2 vs. <2) | 0.64 |
| LDH (normal vs. > normal for age/sex) | 0.48 |
| Performance status (≧2 vs. <2) | 0.09 |
| Stage (I/II vs. III/IV) | 0.88 |
| Number of pre-transplant treatment regimens | 0.13 |
| Pre-mobilization ALC | 0.44 |
| Sex | 0.93 |

### Example 2

### Early NK Cell Engraftment Improves PFS after ASCT

The determine which ALC-15 lymphocyte subset(s) affects survival post ASCT, absolute numbers of T cells, B cells, and NK cells were studied in 29 patients (10 with MM and 19 with NHL) by flow cytometric analyses of peripheral blood specimens on day 15 post-ASCT. At a median follow-up of 16 months (range 2-38 months), 15 patients had evidence of disease relapse or progression, including 7 who died. There were no treatment-related deaths. At day 15 post-ASCT, 15 patients had attained a normal absolute NK count (ANKC; normal rage 80-597), 5 a normal CD8 count, and 2 a normal CD3 count. None of the patients displayed normal numbers of CD4 or CD19 cells. The effect of day 15 ANKC on PFS was analyzed. Table 7 summarizes the median and 2 year PFS based on ALC≧500 cells/μl and ANKC≧80 cells/μl by day 15 after ASCT. Both ACL≧500 cells ml and ANKC≧80 cells/μl were found to be associated with superior PFS. IN the sub-group of patients with ALC<500 cells/μl, patients with ANKC≧80 cells/μl had better PFS compared to those with ANKC<80 cells/μl (p<0.0059). In the sub-group of patients with ALC≧500 cells/μl, only one patient had ANKC<80 cells/μl. These data suggest that ANKC-15 may be more relevant than ALC-15 to the observed clinical benefit post-ASCT.

TABLE 7
PFS based on ALC and NK cell numbers at day 15 after ASCT

| | Median | 2 years | |
|---|---|---|---|
| Lymphocytes | months) | (% PFS) | P-value |
| ALC ≧ 500 cells/μl (n = 11) | Not reached | 83 vs. 18 | p < 0.0078 |
| vs. ALC < 500 cells/μl (n = 18) | vs. 7 | | |
| NK ≧ 80 cells/μl (n = 15) | Not reached | 89 vs. 0 | P < 0.0001 |
| vs. NK < 80 cells/μl (n = 14) | vs. 6 | | |
| ALC < 500 cells/μl (sub-group): | Not reached | 75 vs. 0 | P < 0.0059 |
| NK ≧ 80 cells/μl (n = 5) vs. | vs. 6 | | |
| NK < 80 cells/μl (n = 13) | | | |

### Example 3

### The Number of Re-Infused NK Cells Correlates with ALC Recovery

### Patient Sample

Seven patients (3 multiple myeloma and 4 non-Hodgkin's lymphoma) who were candidates for autologous peripheral stem cell transplantation were entered in the study from October 1999 until April 2000. None of the non-Hodgkin's patients received rituxan therapy.

PBPC Mobilization and Collection:

Non-Hodgkin's lymphoma patients received G-CSF (10 mg/kg) daily for 5-7 consecutive days by subcutaneous injection. Multiple myeloma patients received cyclophosphamide (1.5 g/m²) plus G-CSF (10 mg/kg). Apheresis sessions were started on day 5 of G-CSF administration and were performed with a Fenwal CS3000-plus blood-cell collector (Baxter, Deerfiel, Ill.). Ten to twelve liters of blood were processed daily, at flow rates of 50-60 ml/min using Hickman catheter or antecubital veins. Patients underwent daily apheresis sessions until a target of 2.0×10⁶ CD34 cells/kg or greater was achieved. The median time from collection to sample analysis was 16 months (range 15-17 months).

Immunophenotyping and Flow Cytometry:

Frozen apheresis sample product from each collection was saved for the study. Each sample was thawed in a water bath at 37° C. After thawing, each apheresis sample was labeled with the following monoclonal antibodies (moAB): fluorescein isothiocyanate (FTIC)-conjugated anti-CD3, and anti-CD19; phycoerythrin (PE)-conjugated anti-CD4⁺, anti-CD8⁺, and anti-CD16⁺/CD56⁺, and Simultest Control (IgG1 FTIC [symbol] IgG2a PE), all purchased from Becton Dickinson Immunocytometry Systems (BDIS; San Jose, Calif.). FACS Lysing Solution (BDIS) was used to lyse erythrocytes before staining. Flow cytometry was performed on a FACScan (BDIS) equipped with a 15-mV air-cooled argonion laser tuned at 488 nm. Data were analyzed using the software Lysis II. The percentage of cells labeled with the particular moAB was multiplied by the total WBC/kg to give the total antibody-positive cells/kg in the apheresis product.

Statistics:

The association of day 15 ALC and re-infused autologous graft T cells, B cells, and NK cells from the apheresis product was studied using Spearman rank correlation coefficient.

Results:

The seven patients included in the study had a median age at transplantation of 54 years (range 24-68 years). Table 8 shows the patients' characteristics. Four patients achieved an ALC≧500 cells/ml at day 15 post-ASCT, and only one patient had evidence of relapse. Three patients who achieved an ALC<500 cells/ml at day 15 post-ASCT had relapsed. Two patients required more than three apheresis collections to obtain CD34 count≧2.0×10⁶/kg. T cells and NK cells were the main lymphocyte subsets identified from the apheresis product. The total absolute numbers of T, B and NK cells/kg per patient in the apheresis product and post-ASCT day 15 ALC are shown in Table 8. Mean number of the autologous graft lymphocyte subsets (≧10⁶/kg) for the cohort group were: CD3⁺: 133 (±38), CD4⁺: 46 (±12), CD8⁺: 60 (±131), CD19⁺: 3 (±2), and CD16⁺/CD56⁺/CD32: 47 (±14). As shown in Table 9, NK cells were the only lymphocyte subset from the re-infused autologous graft of all the patients in the study with a strong correlation with ALC-15. There was poor correlation between the CD34 cell dose/kg and ALC at day 15 (r=0:17).

TABLE 8
Post-ASCT ALC-15 and re-infused autologous graft
lymphocyte subsets and patient characteristics

| | | | | Autologous graft | | | | |
|---|---|---|---|---|---|---|---|---|
| | | Time to relapse | | (ALC subsets, cells/kg × 10⁶) | | | | |
| Patient | Disease | (months) | ALC-15* | CD3⁺ | CD4⁺ | CD8⁺ | CD19⁺ | CD16⁺/CD56⁺/CD3⁻ |
| 1 | MM | 11 | 415 | 143 | 110 | 33 | 0.91 | 49 |
| 2 | MM | 14 | 750 | 50.7 | 34.6 | 25.3 | 0.53 | 102.6 |
| 3 | MM | 14 | 480 | 79.3 | 48 | 21 | 0.18 | 26 |
| 4 | NHL | 3 | 175 | 14 | 3.4 | 6.6 | 0.7 | 8.4 |
| 5 | NHL | NR** | 560 | 190 | 47 | 137 | 16 | 53 |
| 6 | NHL | NR | 600 | 277 | 67 | 213 | 0.18 | 49 |
| 7 | NHL | NR | 620 | 218 | 176 | 47.4 | 1.65 | 164 |

*cells/ml
**no relapse

TABLE 9
Correlation of autologous graft lymphocyte
subsets absolute numbers to ALC-15

| | Spearman's | |
|---|---|---|
| Autologous graft | correlation | |
| lymphocyte subset | coefficient (r) | P-value |
| CD3⁺ | 0.21 | 0.64 |
| CD4⁺ | 0.32 | 0.48 |
| CD8⁺ | 0.39 | 0.38 |
| CD19⁺ | 0.14 | 0.76 |
| CD16⁺/CD56⁺/CD3⁻ | 0.77 | 0.04* |

*Statistically significant

### Example 4

### Timely Reconstitution of Immune Competence Affects Clinical Outcome Following ASCT

### Post Transplant ALC Recovery

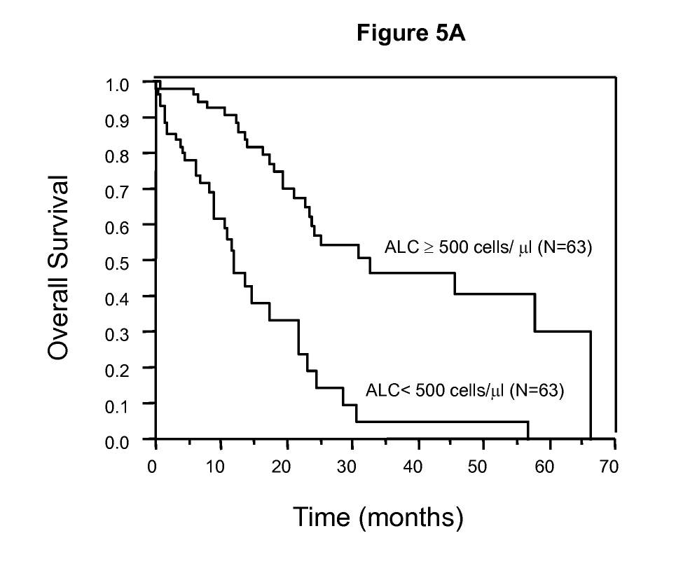

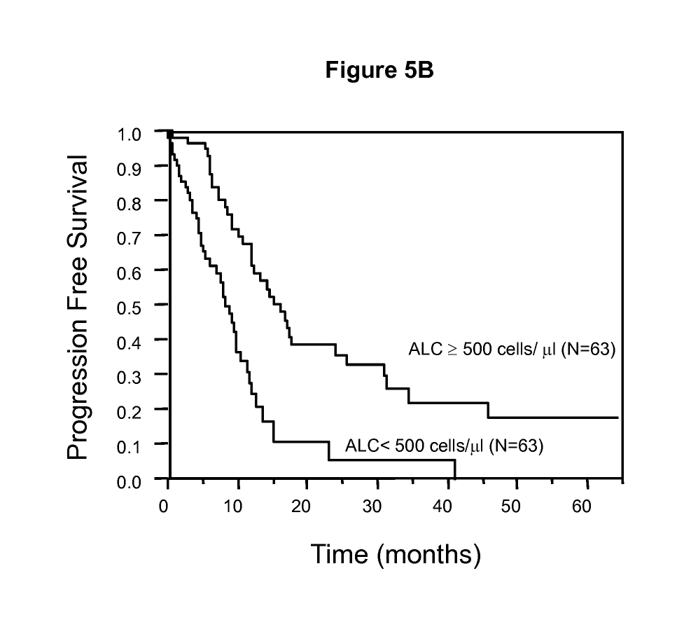

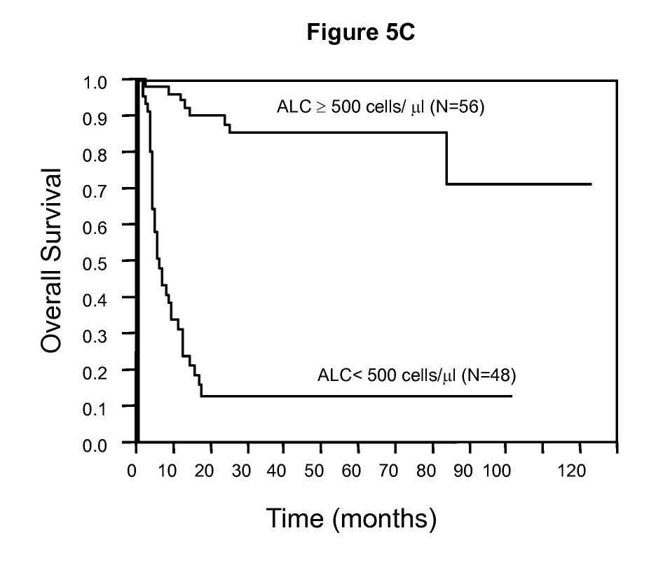

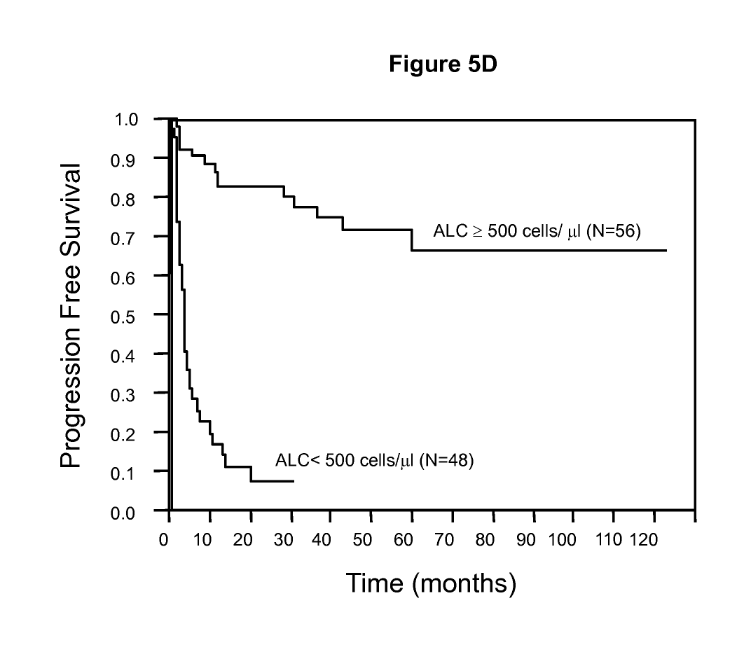

To assess whether early ALC recovery has prognostic significance post-ASCT, ALC at day 15 (ALC-15) was analyzed post-ASCT in MM and NHL patients. The median OS and PFS for the MM group were significantly better for patients with ALC≧500 cells/μl versus ALC<500 cells/μl (OS 33 months vs. 12 months, p<0.0001; PFS 16 months vs. 8 months, p<0.0001; FIGS. 5A and 5B, respectively). For the NHL patients, the median OS and PFS also were significantly better for patients with ALC≧500 cells/μl versus ALC<500 cells/μl (OS not yet reached vs 6 months, p<0.0001; PFS not yet reached vs 4 months, p<0.0001; FIGS. 5C and 5D, respectively). The superior survival observed with early (day 15) ALC≧500 cells/μl recovery in different malignant diseases suggests that the anti-tumor activity of the autologous immune system post-ASCT is not disease specific. However, the fact that none of the patients developed GVHD argues in favor of a possibly more specific immune response against tumor (and not the host) in the post-ASCT setting.

Kinetics of Absolute Lymphocyte Count Recovery Post-ASCT:

A limitation in these initial studies was the selection of a single time point (day 15 post-ASCT) as the only discriminator of clinical outcome in relation to lymphocyte (immune system) recovery. To address this issue, OS and PFS were examined at other time points. These studies demonstrated superior OS and PFS in patients achieving an ALC≧500 cells/μl by day 15 post-ASCT compared with patients achieving an ALC≧500 cells/μl recovery by day 30 (OS not reached vs. 9 months, p<0.0001; PFS 152 vs. 3 months, p<0.000: Yoong et al. (2001) Blood 98(11):abstract. #2889). The worsening OS and PFS with delayed ALC recovery post-ASCT may be explained by the concept of a “tumor burden threshold” effect since, for example, in pre-clinical animal models, the dose of inoculated tumor cells affects the ability of the immune system to eradicate tumor (Ackerstein et al. (1991) Blood 78:1212-1215). In the ASCT setting, the delayed ALC recovery may allow minimal residual disease to outgrow the rate of immune reconstitution, thereby overcoming the benefits of an autologous graft versus tumor (GVT) effect.

Effector Cell Subsets Involved in Early Lymphocyte Recovery:

Relevant effector cells involved in the ALC recovery and their relationship to clinical outcome post-ASCT should fulfill two criteria: 1) normal quantitative recovery, and 2) normal functional activity. To identify the effector cells conveying a better survival using ALC as a surrogate maker of immune recovery post-ASCT, an understanding of immune reconstitution after hematopoietic stem cell transplantation is needed. Although there are similarities in immune reconstitution following Allo-SCT and ASCT, Allo-SCT involves the use of immunosuppressive therapy to control GVHD, which interferes with early developmental stages of immune reconstitution. Because ASCT does not entail development of GVHD or the use of immunosuppressive drugs, it presents a more direct insight into the biology of immune reconstitution following stem cell transplantation.

Immunological reconstitution is a gradual process (Guillaume et al. (1998) Blood 92:1471-1490; and Porrata et al. (2001) Mayo Clin. Proc. 76:407-412). Delayed quantitative and qualitative T and B cell reconstitution is observed from months to years post-ASCT, whereas NK cells recover normal absolute numbers and function much more quickly, as demonstrated herein. In fact, NHL patients achieving normal absolute numbers of NK cells at day 15 post-ASCT have superior median OS and PFS compared with NHL patients with low absolute numbers of NK cells at day 15 post-ASCT (OS not reached vs. 26 months, p<0.0011; PFS not reached vs. 6 months, p<0.0001).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a patient, said method comprising:
a) collecting from said patient a biological sample comprising NK cells;
b) monitoring the number of collected NK cells;
c) repeating steps (a) and (b) until the total number of collected NK cells is at least 0.5×10⁹ cells per kg; and
d) returning said collected NK cells to said patient.

2. The method of claim 1, wherein said biological sample further comprises erythrocytes.

3. The method of claim 2, wherein said method further comprises returning at least 90% of said erythrocytes to said patient.

4. The method of claim 1, further comprising, prior to returning said collected NK cells to said patient, contacting said collected NK cells with one or more agents that stimulate function or activity of NK cells.

5. The method of claim 4, wherein said collected NK cells are retained within a vessel comprising said one or more agents.

6. The method of claim 5, wherein said vessel comprises said one or more agents prior to placement of said NK cells within said vessel.

7. The method of claim 6, wherein said vessel comprises an interior surface, and wherein said one or more agents are dispersed on said interior surface.

8. The method of claim 6, wherein said one or more agents are in the form of a solid.

9. The method of claim 8, wherein said solid is a powder.

10. The method of claim 4, wherein said one or more agents are selected from the group consisting of IL-2, IL-12, IL-15, IL-17, IL-21, IFN-alpha, and IFN-gamma.

11. The method of claim 4, wherein said agent is IL-2.

12. The method of claim 11, wherein said collected NK cells are contacted with IL-2 at a dose of 1.5 to 2.0 million units.

13. The method of claim 1 or claim 4, further comprising, prior to collecting said biological sample, administering to said patient one or more agents that stimulate NK cell function or activity.

14. The method of claim 13, wherein said one or more agents are selected from the group consisting of IL-2, IL-12, IL-15, IL-17, IL-21, IFN-alpha, and IFN-gamma.

15. The method of claim 13, wherein said one or more agent is IL-2.

16. The method of claim 1, further comprising, prior to returning said collected NK cells to said patient, subjecting said patient to an immunosuppressive treatment.

17. The method of claim 16, wherein said immunosuppressive treatment is radiotherapy or chemotherapy.

18. The method of claim 16, wherein said immunosuppressive treatment is surgery with anesthesia.

19. The method of claim 1, wherein said patient is diagnosed with cancer.

20. The method of claim 19, wherein said cancer is breast cancer, non-Hodgkin's lymphoma, multiple myeloma, Hodgkin's disease, or acute myeloid leukemia.

21. The method of claim 19, wherein said cancer is non-Hodgkin's lymphoma.

22. The method of claim 19, wherein prior to collection of said biological sample, said patient is in remission from said cancer.

23. The method of claim 19, wherein prior to return of said collected NK cells, said patient is in remission from said cancer.

24. The method of claim 1, further comprising:
f) monitoring the number of NK cells within said patient; and
g) if said number of NK cells in said patient at day 15 is less than 80 NK cells/microliter, administering to said patient one or more agents selected from the group consisting of IL-2, IL-12, IL-15, IL-17, IL-21, IFN-alpha, and IFN-gamma.

25. The method of claim 1, wherein step (b) further comprises monitoring the number of collected CD34⁺ cells, wherein step (c) further comprises repeating steps (a) and (b) until the total number of collected CD34⁺ cells is at least 2.0×10⁶ cells per kg, and wherein step (d) further comprises returning said collected CD34⁺ cells to said patient.

26. The method of claim 25, further comprising, prior to collecting said biological sample, administering to said patient one or more agents that can (i) stimulate proliferation of stem cells and/or progenitor cells, and/or (ii) stimulate mobilization of stem cells and/or progenitor cells to the peripheral circulation.

27. The method of claim 26, wherein said one or more agents are selected from the group consisting of G-CSF, GM-CSF, SCF, IL-2, IL-7, IL-8, IL-12, and flt-3 ligand.

28. A method for treating a patient, said method comprising:
a) administering autologous lymphocytes to said patient, wherein said autologous lymphocytes are administered in an amount of at least 0.5×10⁹ cells/kg;
b) monitoring the number of NK cells within said patient; and
c) if said number of NK cells at day 15 is less than 80 cells/μL of blood, administering to said patient one or more agents to stimulate NK cell function or activity.

29. The method of claim 28, wherein said autologous lymphocytes are removed from said patient, and wherein, prior to said removal of said autologous lymphocytes, said patient is treated with one or more agents selected from the group consisting of IL-2, IL-12, IL-15, IL-17, IL-21, IFN-alpha, and IFN-gamma.

30. The method of claim 28 wherein, prior to said administering to said patient, said autologous lymphocytes are contacted in vitro with one or more agents selected from the group consisting of IL-2, IL-12, IL-15, IL-17, IL-21, IFN-alpha, and IFN-gamma.

31. The method of claim 28, wherein said patient is diagnosed with cancer.

32. The method of claim 31, wherein said cancer is breast cancer, non-Hodgkin's lymphoma, Hodgkin's disease, multiple myeloma, or acute myeloid leukemia.

33. The method of claim 31, wherein said cancer is non-Hodgkin's lymphoma.

34. A method for obtaining a population of lymphocytes, said method comprising:
a) collecting from a subject a biological sample comprising lymphocytes;
b) monitoring the number of NK cells within the collected lymphocytes; and
c) repeating steps (a) and (b) until the total number of NK cells collected from said subject is at least 0.5×10⁹ cells/kg.

35. The method of claim 34, further comprising retaining said collected lymphocytes within a vessel that comprises an identifier corresponding to said subject, and contacting said collected lymphocytes with one or more agents that stimulate NK cell function or activity.

36. The method of claim 35, wherein said one or more agents are selected from the group consisting of IL-2, IL-12, IL-15, IL-17, IL-21, IFN-alpha, and IFN-gamma.

37. The method of claim 34, further comprising, prior to collecting said biological sample from said subject, administering to said subject one or more agents to stimulate NK cell function or activity.

38. The method of claim 37, wherein said one or more agents are selected from the group consisting of IL-2, IL-12, IL-15, IL-17, IL-21, IFN-alpha, and IFN-gamma.

39. A container comprising a population of lymphocytes removed from a subject, wherein said population comprises an amount of NK cells that is at least 0.5×10⁹ cells/kg, and wherein said container comprises an identifier corresponding to said subject.

40. The container of claim 39, wherein said container is a blood bag.

41. The container of claim 39, further comprising one or more agents that stimulate NK cell function or activity.

42. A container comprising an inner surface, wherein one or more agents are dispersed on said inner surface, and wherein said one or more agents stimulate NK cell function or activity.

43. The container of claim 42, wherein said one or more agents are selected from the group consisting of IL-2, IL-12, IL-15, IL-17, IL-21, IFN-alpha, and IFN-gamma.
